(19) United States
(12) Patent Application Publication
(10) Pub. No.: US 2011/0196383 A1
(43) Pub. Date: Aug. 11, 2011

(54) Atherosclerotic Plaque Dissolution Composition

(75) Inventors: Kusai Saadeldin Aziz, Visalia, CA (US); Gary Michael Saxton, Sunnyvale, CA (US)

(73) Assignee: Atherolysis Medical, Inc

(21) Appl. No.: 13/087,520

(22) Filed: Apr. 15, 2011

Related U.S. Application Data
(63) Continuation-in-part of application No. 12/773,028, filed on May 4, 2010.
(60) Provisional application No. 61/175,458, filed on May 5, 2009.

(51) Int. Cl. A61B 17/22; A61K 31/015; A61K 31/047; A61K 31/20; A61K 31/01; A61K 31/616; A61K 31/19; A61K 31/201; A61K 31/045; A61K 31/397; A61K 31/505; A61K 31/05; A61K 31/02; A61K 33/04; A61K 31/145; A61K 31/7076; A61K 31/277; A61K 38/16; A61P 9/00

(52) U.S. Cl. 606/127; 514/763; 514/738; 514/558; 514/762; 514/165; 514/557; 514/560; 514/724; 514/210.02; 514/275; 514/733; 514/758; 424/701; 514/642; 514/46; 514/523; 514/164; 514/161; 514/1.1

(57) ABSTRACT

A method and chemical composition for modifying and dissolving atherosclerotic plaques formed in a patient's blood vessels is provided. A chemical composition comprising one or more of an organic substance, an inorganic substance, and a bioactive substance is administered at sites of the plaques. The chemical composition comprises one or more of d-limonene, propylene glycol, octanoic acid, 2-octane, glycerine, acetylsalicylic acid, acetic acid, omega-3 fatty acids, ethanol, methanol, ezetimibe, rosuvastatin, resveratrol, lactic acid, gluconic acid, chloroform, carbon disulfide, dichloromethane, toluene, lauryldimethyl hydroxysultaine, and any combination thereof. The chemical composition enables modification of plaques by altering their composition. The modification comprises partial dissolution, complete dissolution, or elimination of the plaques, and makes the plaques amenable to different forms of plaque treatment. The modified plaques are eliminated from the patient's body. The modification or elimination of the plaques facilitates treatment of cardiovascular diseases caused due to plaques formed in the blood vessels.

### CROSS REFERENCE TO RELATED APPLICATIONS

This application is a continuation-in-part application of non-provisional patent application Ser. No. 12/773,028 titled “Atherosclerotic Plaque Dissolution Composition”, filed on May 4, 2010 in the United States Patent and Trademark Office, which claims the benefit of provisional patent application No. 61/175,458 titled “Atherosclerotic Plaque Dissolution Composition”, filed on May 5, 2009 in the United States Patent and Trademark Office.

The specifications of the above referenced patent applications are incorporated herein by reference in their entirety.

### BACKGROUND

The method and composition disclosed herein, in general, relates to plaque removal. More particularly, the method and composition disclosed herein relates to modifying, dissolving or eliminating plaques formed in blood vessels of a patient for treatment of cardiovascular diseases.

Atherosclerosis and plaque formation in blood vessels such as human arteries, which cause cardiovascular diseases, for example, coronary artery disease, myocardial infarction, carotid artery disease, strokes, and peripheral arterial disease are a common cause of human impairment and death.

Different treatment modalities, for example, balloon angioplasty, stents, atherectomy, and bypass surgery, have been developed to treat blockages caused by plaques. All these modalities are based on physical changes such as opening of a focal blockage or bypassing the blockages. However, these modalities do not address the pathological process of elimination of the plaques formed in the blood vessels.

Different physical methods typically used for treating plaques lead to the risk of adverse events such as death of the patient. For example, stents can lead to perforation of arteries and have certain incidences of restenosis of the arteries and thrombosis leading to myocardial infarctions and possibly death. Moreover, stents require use of blood thinners and increase the risk of bleeding.

Chemical dissolution methods have been used in dissolving gall bladder stones composed mainly of cholesterol but have not been implemented in atherosclerotic plaque dissolution.

Hence, there is long felt but unresolved need for a method and a chemical composition that dissolves atherosclerotic plaques for treating cardiovascular diseases by eliminating plaques formed in the blood vessels of the patient suffering from cardiovascular diseases or by modifying plaque characteristics to make the plaques more amenable to other physical methods of plaque treatment such as balloon angioplasty or stenting.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The method and chemical composition disclosed herein address the above stated need for treating cardiovascular diseases by modifying, dissolving, and eliminating plaques formed in blood vessels of a patient suffering from cardiovascular diseases. A chemical composition comprising variable percentages of one or more of an organic substance, an inorganic substance, and a bioactive substance is administered at sites of the plaques formed in the blood vessels of the patient. The chemical composition can comprise only an “organic substance”, or only an “inorganic substance”, or only a “bioactive substance”; or a combination of two or more of these constituents. The chemical composition is administered at the sites of plaque formation, for example, through a catheter. The chemical composition comprises, for example, one or more of about 0.1% to about 99.9% by weight of d-limonene, about 0.1% to about 99.9% by weight of propylene glycol, about 0.1% to about 99.9% by weight of octanoic acid, about 0.1% to about 99.9% by weight of 2-octane, about 0.1% to about 99.9% by weight of glycerine, about 0.1% to about 99.9% by weight of acetylsalicylic acid, about 0.1% to about 99.9% by weight of acetic acid, about 0.1% to about 99.9% by weight of omega-3 fatty acids, about 0.1% to about 99.9% by weight of ezetimibe, about 0.1% to about 99.9% by weight of rosuvastatin, about 0.1% to about 99.9% by weight of cis-resveratrol and/or trans-resveratrol, about 0.1% to about 99.9% by weight of lactic acid, about 0.1% to about 99.9% by weight of gluconic acid, about 0.1% to about 99.9% by weight of chloroform, about 0.1% to about 99.9% by weight of carbon disulfide, about 0.1% to about 99.9% by weight of dichloromethane, about 0.1% to about 99.9% by weight of toluene, about 0.1% to about 99.9% by weight of lauryldimethyl hydroxysultaine, and any combination thereof with a sufficient amount of a base composition to adjust the total weight percentage of the chemical composition to 100%. The base composition comprises, for example, water or a physiologic solution such as a saline solution, dextrose water, an organic solvent, etc.

In an embodiment, one or more additives may be added to the chemical composition disclosed herein. The additives are selected from a group comprising, for example, monoglyceride, monoctanoin, diethyl ether, halothane, ethanol, methanol, steroids, folic acid, heparin, octanediol, adenosine, high density lipoproteins (HDL), oils, etc., and any combination thereof. The chemical composition disclosed herein further comprises one or more optional medications, for example, direct thrombin inhibitors, thrombolytics, statins, antiplatelets, calcium channel blockers such as verapamil, etc., and any combination thereof. Each of the constituents of the chemical composition disclosed herein can be used independently or in combination with one or more of the other constituents disclosed herein for modifying, dissolving, and eliminating plaques formed in the blood vessels of the patient.

The chemical composition enables modification of the plaques by altering composition of the plaques in the blood vessels and thereby facilitating the removal of the modified plaques. The modification comprises partial dissolution, complete dissolution or elimination of the plaques. The modification makes the plaques amenable to different forms of plaque treatment, for example, balloon angioplasty, stenting, atherectomy, etc. The modified plaques are eliminated from the body of the patient. The modification or elimination of the plaques facilitates treatment of the cardiovascular diseases caused due to the plaques formed in the blood vessels of the patient.

The administered chemical composition is retained at the sites of plaque formation for a predetermined period of time to enable the modification of the plaques before removing the administered chemical composition by suctioning or flushing. The process of administering and removing the chemical composition may be repeated multiple times or may be continuous in nature, wherein the chemical composition is continuously delivered to the delivery site and at the same time suctioned or aspirated from such site. The chemical composition disclosed herein can be administered for plaque dissolution in different arterial blood vessels, for example, coronary arteries, peripheral arteries, arteries in lower extremities of the patient's body, renal arteries, carotid arteries, cerebral vessels, etc. The modification of the plaques by the administered chemical composition comprises partial dissolution, complete dissolution, or modification of the plaques in the coronary arteries, the peripheral arteries, the arteries in the lower extremities of the patient's body, the renal arteries, the carotid arteries, and the cerebral vessels for enhancing luminal patency of these arterial blood vessels. The modification of the plaques by the administered chemical composition also comprises partial dissolution of the plaques for creating a channel for blood flow and for passing interventional equipment for different forms of plaque treatment.

In an embodiment, adjunctive methods are administered along with the chemical composition to obtain desired modification of the plaques. The adjunctive methods comprise, for example, utilizing one or more of low and high temperatures, pressure, radio magnetic waves, physical agitation, etc.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1 illustrates a method for modifying and dissolving plaques formed in blood vessels of a patient.

FIG. 2 exemplarily illustrates a process flow for chemical dissolution of plaques formed in blood vessels of a patient.

### DETAILED DESCRIPTION OF THE INVENTION

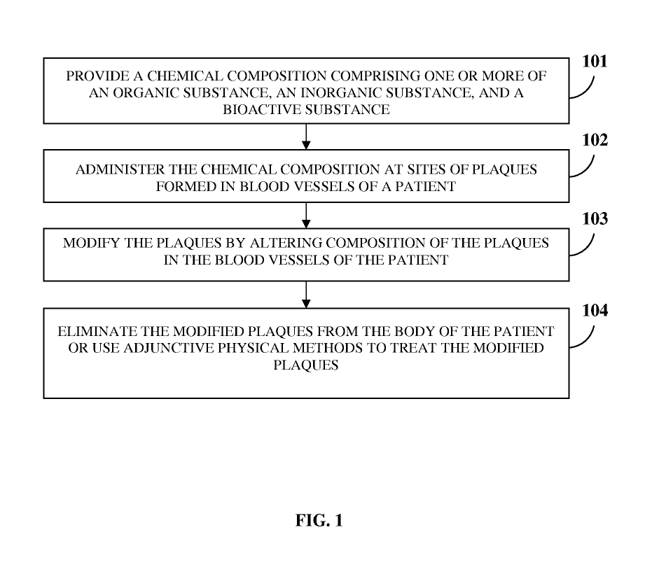

FIG. 1 illustrates a method for modifying and dissolving plaques formed in blood vessels of a patient. The method disclosed herein is used for treating cardiovascular diseases caused due to plaques formed in the blood vessels of the patient. Cardiovascular diseases are a class of diseases related to the heart or the blood vessels. The plaques are formed, for example, due to high levels of cholesterol in blood plasma. The plaques are also formed due to calcification. Calcification is the process of hardening of the tissues due to accumulation of mineral calcium in the tissues. Thrombosis is a pathological condition in which blood clots are formed within the blood vessels resulting in plaques. The plaques are, for example, atherosclerotic plaques. The atherosclerotic plaques are multiple plaques formed within the arteries.

A chemical composition, also referred to as a plaque dissolution composition, comprising one or more of an organic substance, an inorganic substance, and a bioactive substance is provided 101. The chemical composition can comprise only an “organic substance”, or only an “inorganic substance”, or only a “bioactive substance”; or a combination of two or more of these constituents. The bioactive substance is, for example, a substance that reacts with cells or tissues of a living body. A substance which is in a form that cells or tissues of a living body can easily absorb and utilize is referred to as a bioactive substance. One or more of the organic substance, the inorganic substance, and the bioactive substance are selected from a group comprising one or more of d-limonene, propylene glycol, octanoic acid, 2-octane, glycerine, acetylsalicylic acid, acetic acid, omega-3 fatty acid, ethanol, methanol, ezetimibe, rosuvastatin, resveratrol, lactic acid, gluconic acid, chloroform, carbon disulfide, dichloromethane, toluene, lauryldimethyl hydroxysultaine, etc., and any combination thereof. The chemical composition disclosed herein is, for example, composed of a single chemical or multiple chemicals.

The chemical composition disclosed herein comprises, for example, one or more of about 0.1% to about 99.9% by weight of d-limonene, about 0.1% to about 99.9% by weight of propylene glycol, about 0.1% to about 99.9% by weight of octanoic acid, about 0.1% to about 99.9% by weight of 2-octane, about 0.1% to about 99.9% by weight of glycerine, about 0.1% to about 99.9% by weight of acetylsalicylic acid, about 0.1% to about 99.9% by weight of acetic acid, about 0.1% to about 99.9% by weight of omega-3 fatty acid, about 0.1% to about 99.9% by weight of ezetimibe, about 0.1% to about 99.9% by weight of rosuvastatin, about 0.1% to about 99.9% by weight of cis-resveratrol and/or trans-resveratrol, about 0.1% to about 99.9% by weight of lactic acid, about 0.1% to about 99.9% by weight of gluconic acid, about 0.1% to about 99.9% by weight of chloroform, about 0.1% to about 99.9% by weight of carbon disulfide, about 0.1% to about 99.9% by weight of dichloromethane, about 0.1% to about 99.9% by weight of toluene, about 0.1% to about 99.9% by weight of lauryldimethyl hydroxysultaine, and any combination thereof with a sufficient amount of an optional base composition to adjust the total weight percentage of the chemical composition to 100%. The base composition comprises, for example, water or a physiologic solution such as a saline solution, dextrose water, an organic solvent, etc., or any combination thereof.

D-limonene is a liquid hydrocarbon classified as a cyclic terpene possessing a lemon like fragrance. D-limonene increases the rate of solubility of plaques over a predetermined period of time. The chemical composition disclosed herein comprises d-limonene dissolved in water or an organic solvent in a concentration of about 0.1% to about 99.9%. In an example, a concentration of about 98% of d-limonene in the chemical composition disclosed herein showed effectiveness in dissolving atherosclerotic plaque.

Propylene glycol is a colorless, nearly odorless, clear, viscous liquid with a faintly sweet taste, hygroscopic and miscible with water, acetone, and chloroform. The chemical composition disclosed herein comprises propylene glycol dissolved in water or an organic solvent in a concentration of about 0.1% to about 99.9%. In an example, a concentration of about 99.9% of propylene glycol in the chemical composition disclosed herein showed effectiveness in dissolving atherosclerotic plaque.

Octanoic acid, also known as caprylic acid, is an eight-carbon saturated fatty acid. Octanoic acid is found naturally in the milk of various mammals. The chemical composition disclosed herein comprises octanoic acid dissolved in water or an organic solvent in a concentration of about 0.1% to about 99.9%. In an example, a concentration of about 98% of octanoic acid in the chemical composition disclosed herein showed effectiveness in dissolving atherosclerotic plaque.

The chemical composition disclosed herein comprises 2-octane dissolved in water or an organic solvent in a concentration of about 0.1% to about 99.9%. In an example, a concentration of about 98% of 2-octane in the chemical composition disclosed herein showed effectiveness in dissolving atherosclerotic plaque.

Glycerine is a polyol compound. Glycerine is a colorless, odorless, viscous liquid that is used in pharmaceutical formulations. Glycerine has three hydrophilic hydroxyl groups that are responsible for its solubility in water and its hygroscopic nature. Glycerine increases the rate of solubility of plaques over a predetermined period of time. The chemical composition disclosed herein comprises glycerine dissolved in water or an organic solvent in a concentration of about 0.1% to about 100%. In an example, a concentration of about 100% of glycerine in the chemical composition disclosed herein showed effectiveness in dissolving atherosclerotic plaque.

Omega-3 fatty acids are a family of unsaturated fatty acids orally known to reduce the risk of coronary artery disease. The chemical composition disclosed herein comprises omega 3 fatty acids dissolved in water or an organic solvent in a concentration of about 0.1% to about 99.9%. In an example, a concentration of about 82% of omega-3 fatty acids in the chemical composition disclosed herein showed effectiveness in dissolving atherosclerotic plaque.

Acetylsalicylic acid, also known as aspirin, is known to have anti-platelet activity and is therefore used to treat and prevent heart attack. The chemical composition disclosed herein comprises acetylsalicylic acid dissolved in water or an organic solvent in a concentration of about 0.1% to about 99.9%. In an example, a concentration of about 12% of acetylsalicylic acid in the chemical composition disclosed herein showed effectiveness in dissolving atherosclerotic plaque.

The chemical composition disclosed herein comprises methanol dissolved in water or an organic solvent in a concentration of about 0.1% to about 99.9%. In an example, a concentration of about 99.9% of methanol in the chemical composition disclosed herein showed effectiveness in dissolving atherosclerotic plaque.

The chemical composition disclosed herein comprises ethanol dissolved in water or an organic solvent in a concentration of about 0.1% to about 99.9%. In an example, ethanol at a concentration of about 94% to about 96% showed effectiveness in dissolving atherosclerotic plaque.

Ezetimibe is used to treat high cholesterol by preventing gastrointestinal absorption. In an example, the chemical composition disclosed herein comprises ezetimibe dissolved in water or an organic solvent in a concentration of about 0.1% to about 99.9%.

Rosuvastatin is a statin used to treat high cholesterol and related conditions and to prevent cardiovascular disease. In an example, the chemical composition disclosed herein comprises rosuvastatin dissolved in water or an organic solvent in a concentration of about 0.1% to about 99.9%.

Resveratrol, also known as 3,5,4′-trihydroxy-trans-stilbene, is a stilbenoid, a type of natural phenol. Resveratrol is found in the skin of red grapes and in other fruits. Red wine contains a high level of resveratrol and some scientists believe resveratrol is one of the factors behind the French paradox, while others believe proanthocyanidin is the active ingredient involved. Resveratrol has also been produced by chemical synthesis or by biotechnological synthesis, that is, metabolic engineered microorganisms and is sold as a nutritional supplement derived primarily from Japanese knotweed. Resveratrol exists as two geometric isomers: cis-resveratrol and trans-resveratrol. Trans-resveratrol and cis-resveratrol can be either free or bound to glucose. In an example, the chemical composition disclosed herein that comprises resveratrol dissolved in water or an organic solvent in a concentration of about 0.1% to about 99.9%, showed effectiveness in dissolving atherosclerotic plaque.

Lactic acid is a chemical produced in the human body and has detergent like abilities. Lactic acid is also present in milk. In an example, the chemical composition disclosed herein comprises lactic acid dissolved in water or an organic solvent in a concentration of about 0.1% to about 99.9%.

Gluconic acid is present naturally in fruit and honey. In an example, the chemical composition disclosed herein comprises gluconic acid dissolved in water or an organic solvent in a concentration of about 0.1% to about 99.9%.

Chloroform is a colorless, dense liquid having a sweet fragrance. The chemical composition disclosed herein comprises chloroform dissolved in water or an organic solvent in a concentration of about 0.1% to about 99.9%. In an example, a concentration of about 95% of chloroform in the chemical composition disclosed herein showed effectiveness in dissolving atherosclerotic plaque.

Carbon disulfide is a colorless volatile liquid. In an example, a concentration of about 90% of carbon disulfide in the chemical composition disclosed herein showed effectiveness in dissolving atherosclerotic plaque.

Dichloromethane is a colorless, volatile liquid with a moderately sweet aroma. In an example, a concentration of about 92% of dichloromethane in the chemical composition disclosed herein showed effectiveness in dissolving atherosclerotic plaque.

Toluene is a clear, water-insoluble liquid. In an example, a concentration of about 40% of toluene in the chemical composition disclosed herein showed effectiveness in dissolving atherosclerotic plaque.

Lauryldimethyl hydroxysultaine is effective against calcifications. In an example, the chemical composition disclosed herein comprises lauryldimethyl hydroxysultaine dissolved in water or an organic solvent in a concentration of about 0.1% to about 99.9%.

Acetic acid, also known as ethanoic acid, is present in vinegar and used topically to treat skin. Acetic acid can be effective against plaque calcifications. In an example, the chemical composition disclosed herein comprises acetic acid dissolved in water or an organic solvent in a concentration of about 0.1% to about 99.9%.

In an embodiment, the chemical composition for modifying, dissolving, and eliminating plaques disclosed herein further comprises one or more additives for enhancing the function of the chemical composition. The additives are selected from a group comprising, for example, about 0.1% to about 99.9% by weight of d-limonene, about 0.1% to about 99.9% by weight of monoglyceride, about 0.1% to about 99.9% by weight of monoctanoin, about 0.1% to about 99.9% by weight of diethyl ether, about 0.1% to about 99.9% by weight of halothane, about 0.1% to about 99.9% by weight of ethanol, about 0.1% to about 99.9% by weight of methanol, about 5 milligrams (mg) to about 200 mg of steroids, about 1 mg to about 50 mg of folic acid, about 100 units to about 10000 units heparin, about 0.1% to about 99.9% by weight of octanediol, about 1 mg to about 50 mg of adenosine, about 0.1% to about 99.9% by weight of high density lipoproteins (HDL), about 0.1% to about 99.9% by weight of oils, etc., and any combination thereof. The chemical composition further comprises one or more optional medications, for example, direct thrombin inhibitors, thrombolytics, statins, antiplatelets, calcium channel blockers such as verapamil, etc., and any combination thereof. Different constituents of the chemical composition are directed towards different components of the plaques, for example, cholesterol, fibrous tissue, calcification, and thrombosis.

Each of the constituents of the chemical composition disclosed herein can be used independently or in combination with one or more of the other constituents disclosed herein for modifying, dissolving, and eliminating plaques formed in the blood vessels of the patient.

The physical nature of the chemical composition is generally liquid. However, the chemical composition may also contain certain gas components when administered into certain vascular beds such as the arteries in the lower extremities. The chemical composition disclosed herein is administered 102 at sites of the plaques formed in the blood vessels of the patient. The chemical composition is administered, for example, using delivery catheters to the sites of the plaques through the lumen of the artery to the inner surface or the luminal surface of the plaque. Catheters are tubes that can be inserted into a body cavity, for example, the blood vessels. The chemical composition is left in contact with the plaques for a certain period of time to exert chemical action on the plaques.

The administered chemical composition modifies 103 the plaques by altering composition of the plaques formed in the blood vessels of the patient. The chemical composition can be administered to selectively alter composition of the plaques formed in the blood vessels of the patient. The modification comprises partial dissolution, complete dissolution or elimination of the plaques. The modification makes the plaques amenable to different forms of plaque removal treatment, for example, balloon angioplasty, stenting, atherectomy, etc. Adjunctive methods, for example, heat, cold, low and high temperatures, pressure, radio magnetic waves, physical agitation, etc., and any combination thereof are used any time before, during or after administration of the chemical composition disclosed herein to obtain desired modification of the plaques formed in the blood vessels of the patient. The chemical composition alters the plaque characteristic, that is, softens the plaque, when administered to the site of the plaques and hence is used as an adjunctive method for available interventional methods such as balloon angioplasty, stenting, and atherectomy. In an embodiment, the chemical composition disclosed herein is also used for treating chronic total occlusions of arteries in addition to variable degrees of atherosclerosis and luminal narrowing without chronic total occlusion. Chronic total occlusions are a severe form of atherosclerosis with total occlusion of the lumen of the artery. The chemical composition disclosed herein dissolves some of the atherosclerotic plaques and creates a channel for blood flow and for passing interventional equipment for different forms of plaque treatment, for example, balloon angioplasty, stenting, atherectomy, etc.

Furthermore, flushing or suction is used to remove pieces of the plaques. The process of administering and removing the chemical composition disclosed herein may be repeated multiple times or may be continuous in nature, wherein the chemical composition is continuously delivered to the delivery site and at the same time suctioned or aspirated from such site. Alternating flushing and suctioning may also be performed to maintain a constant exposure of the chemical composition disclosed herein to the luminal surface of the plaque by removing atherosclerotic materials that have already undergone alteration mediated by the chemical composition, and replacing the used chemical composition with fresh chemical composition in its original state.

The modified plaques are eliminated 104 from the body of the patient by a process of metabolism followed by excretion from the patient's body. The modified plaques are metabolized and excreted out of the body through different mechanisms and organs such as the liver and kidneys. The dissolution and purging of the plaque substance formed in the blood vessels widens the lumens of the treated arteries and therefore enables treatment of coronary artery disease, peripheral vascular disease, renal artery disease, etc. In an embodiment, adjunctive physical methods may be used 104 to treat the blood vessels having the modified plaques. The modification or elimination of the plaques formed in the blood vessels facilitates treatment of cardiovascular diseases caused due to the plaques formed in the blood vessels.

Balloon angioplasty involves mechanical widening of the blood vessels comprising plaques. Stenting is a procedure that involves use of a stent to open the arteries post angioplasty. Atherectomy is a process of reconstruction of the arteries after surgical removal of the plaques in the arteries. Filter wires, for example, are used in conjugation with the chemical composition disclosed herein to prevent distal embolization. Distal embolization is blockage of a blood vessel due to migration of a solid structure from one point of the blood vessel to another point in a direction of the blood stream.

The chemical composition disclosed herein facilitates partial dissolution, complete dissolution or modification of the plaques in the blood vessels of the heart muscle, that is, in the coronary arteries and enhances luminal patency of the coronary arteries, thereby decreasing incidence and severity of angina or myocardial infarctions also known as heart attacks.

Furthermore, the chemical composition disclosed herein facilitates partial dissolution, complete dissolution, or modification of the plaques in peripheral arteries, arteries in the lower extremities such as legs and feet of the patient's body, and enhances luminal patency of the arteries, thereby decreasing incidence and severity of peripheral vascular disease caused due to formation of plaques in the arteries of arms and legs, limbs ischemia, gangrene, etc. Limbs ischemia is the restricted flow of blood to the limbs due to formation of plaques in the peripheral arteries. The chemical composition disclosed herein also facilitates partial dissolution, complete dissolution, or modification of the plaques in renal arteries and enhances luminal patency of the renal arteries, thereby decreasing incidence of different types of renal failure caused by reduced blood flow. Furthermore, the chemical composition disclosed herein facilitates partial dissolution, complete dissolution, or modification of the plaques in carotid arteries and other cerebral vessels and enhances luminal patency of the carotid arteries and other cerebral vessels, thereby decreasing incidence of certain types of strokes and certain neurological symptoms such as dizziness.

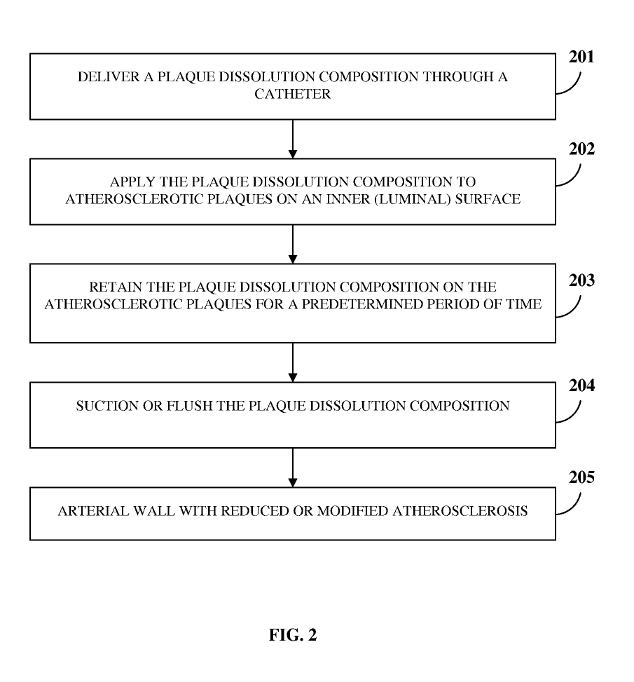

FIG. 2 exemplarily illustrates a process flow for chemical dissolution of plaques formed in blood vessels of a patient. The chemical composition herein referred to as a “plaque dissolution composition”, as disclosed in the detailed description of FIG. 1, is delivered 201 into the lumen of the affected blood vessels through a catheter. The plaque dissolution composition is applied 202 to the atherosclerotic plaques on the inner surface or the luminal surface within the blood vessels. The plaque dissolution composition is retained 203 on the atherosclerotic plaques for a predetermined period of time, for example, in a range of seconds or minutes to react with the atherosclerotic plaques. The plaque dissolution composition, including any dissolved plaques is removed from the affected sites of the arteries by suction or flushing 204 of the plaque dissolution composition. The plaque dissolution composition administered according to the method disclosed herein reduces or modifies 205 atherosclerosis of the arterial walls, and renders the atherosclerotic plaques amenable to other forms of plaque treatment.

The plaque dissolution composition and concentration is selected from biologically acceptable compounds or modified or controlled to be safe to biological tissues. Moreover, thorough testing is performed at different stages to obtain optimal compositions of the plaque dissolution composition. Research is performed by in-vitro testing of different concentrations of the above mentioned chemical compounds individually or in combination, followed by animal models prior to administering the plaque dissolution composition to humans. Furthermore, more than one set of the plaque dissolution composition is developed for different cases of cardiovascular diseases based on different factors, for example, vascular area, type of plaques, etc., according to the degree of calcification, and other characteristics of the patient. Several other chemical compositions can be used for the dissolution or elimination of the plaques according to the method disclosed herein, individually or in combinations with the above mentioned chemical substances and products.

### Example 1

In an ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 98% by weight of d-limonene and about 2% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 2

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 99.9% by weight of propylene glycol and about 0.1% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 3

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 98% by weight of octanoic acid and about 2% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 4

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 98% by weight of 2-octane and about 2% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 5

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 100% by weight of glycerine with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 6

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 12% by weight of acetylsalicylic acid and about 88% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 7

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 82% by weight of omega-3 fatty acids and about 18% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 8

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 20% by weight of ezetimibe and about 80% by weight of a base composition such as normal saline with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 9

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 20% by weight of rosuvastatin and about 80% by weight of a base composition such as normal saline with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 10

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 98% by weight of lactic acid and about 2% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 11

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 80% by weight of gluconic acid and about 20% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 12

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 95% by weight of chloroform and about 5% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 13

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 90% by weight of carbon disulfide and about 10% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 14

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 92% by weight of dichloromethane with methanol and about 8% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 15

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 99.1% by weight of dichloromethane without methanol and about 0.9% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 16

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 40% by weight of toluene and about 60% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 17

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 90% by weight of lauryldimethyl hydroxysultaine and about 10% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 18

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 94% by weight of ethanol and about 6% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 19

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 99.9% by weight of methanol and about 0.1% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 20

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 2.5% by weight of trans-resveratrol and about 97% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 21

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 2.5% by weight of cis-resveratrol and about 97% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 22

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 2.5% by weight of trans-resveratrol, about 2.5% by weight of cis-resveratrol, and about 95% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 23

In another ex vivo experiment on an atherosclerotic cadaveric superficial femoral artery of a human, dissolution of atherosclerotic plaque was noted after exposing the atherosclerotic plaque for about ten minutes to about thirty minutes to the chemical composition comprising about 50% by weight of acetic acid and about 50% by weight of a base composition with the total weight percentage of the chemical composition adjusted to 100% by addition of a sufficient amount of an optional base composition, for example, another chemical compound, an organic solvent, water, a saline solution, dextrose water, etc., or any combination thereof to the chemical composition.

### Example 24

Another Example is a chemical composition comprising about 20% by weight of d-limonene, about 20% by weight of omega 3 fatty acids, about 20% by weight of acetylsalicylic acid, and about 20% by weight of glycerine, with the total weight percentage of the chemical composition adjusted to 100% by addition of about 20% by weight of a base composition, for example, another chemical compound, an organic solvent, water, a saline solution, dextrose water, etc., or any combination thereof to the chemical composition.

### Example 25

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, and about 1% to about 95% by weight of acetylsalicylic acid with the total weight percentage of the chemical composition adjusted to 100% by addition by a base composition, for example, water to the chemical composition.

### Example 26

Another Example is a composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, and about 1% to about 95% by weight of omega-3 fatty acids with the total weight percentage of the chemical composition adjusted to 100% by addition of a base composition, for example, water to the chemical composition.

### Example 27

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, and about 1% to about 95% by weight of ethanol with the total weight percentage of the chemical composition adjusted to 100% by addition of a base composition, for example, water to the chemical composition.

### Example 28

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, and about 1% to about 95% by weight of ezetimibe with the total weight percentage of the chemical composition adjusted to 100% by addition of a base composition, for example, water to the chemical composition.

### Example 29

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, about 1% to about 95% by weight of lactic acid, and about 1% to about 95% by weight of gluconic acid with the total weight percentage of the chemical composition adjusted to 100% by addition of a base composition, for example, water to the chemical composition.

### Example 30

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, about 1% to about 95% by weight of chloroform, and about 1% to about 95% by weight of carbon disulfide with the total weight percentage of the chemical composition adjusted to 100% by addition of a base composition, for example, water to the chemical composition.

### Example 31

Another Example is a chemical composition comprising about 1% to about 95% by weight of d-limonene, about 1% to about 95% by weight of propylene glycol, about 1% to about 95% by weight of octanoic acid, about 1% to about 95% by weight of 2-octane, about 1% to about 95% by weight of glycerine, about 1% to about 95% by weight of dichloromethane, about 1% to about 95% by weight of methanol, about 1% to about 95% by weight of toluene, and about 1% to about 95% by weight of lauryldimethyl hydroxysultaine with the total weight percentage of the chemical composition adjusted to 100% by addition of the chemical composition by a base composition, for example, water to the chemical composition.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

We claim:

1. A method for modifying and dissolving plaques formed in blood vessels of a patient, comprising:
providing a chemical composition comprising one or more of an organic substance, an inorganic substance, and a bioactive substance, wherein said organic substance, said inorganic substance, and said bioactive substance comprise one or more of d-limonene, propylene glycol, octanoic acid, 2-octane, glycerine, acetylsalicylic acid, acetic acid, omega-3 fatty acid, ethanol, methanol, ezetimibe, rosuvastatin, resveratrol, lactic acid, gluconic acid, chloroform, carbon disulfide, dichloromethane, toluene, lauryldimethyl hydroxysultaine, and any combination thereof;
administering said chemical composition at sites of said plaques formed in said blood vessels of said patient; and
modifying said plaques by altering composition of said plaques in said blood vessels by said administered chemical composition, wherein said modification comprises one of partial dissolution, complete dissolution and elimination of said plaques, wherein said modification makes said plaques amenable to different forms of plaque treatment.

2. The method of claim 1, wherein said chemical composition comprises one or more of:
about 0.1% to about 99.9% by weight of said d-limonene;
about 0.1% to about 99.9% by weight of said propylene glycol;
about 0.1% to about 99.9% by weight of said octanoic acid;
about 0.1% to about 99.9% by weight of said 2-octane;
about 0.1% to about 99.9% by weight of said glycerine; and
a sufficient amount of a base composition to adjust total weight percentage of said chemical composition to 100%.

3. The method of claim 1, wherein said chemical composition comprises one or more of:
about 0.1% to about 99.9% by weight of said acetylsalicylic acid;
about 0.1% to about 99.9% by weight of said omega-3 fatty acid;
about 0.1% to about 99.9% by weight of said ezetimibe;
about 0.1% to about 99.9% by weight of said rosuvastatin;
about 0.1% to about 99.9% by weight of said lactic acid;
about 0.1% to about 99.9% by weight of said gluconic acid;
about 0.1% to about 99.9% by weight of said chloroform;
about 0.1% to about 99.9% by weight of said carbon disulfide;
about 0.1% to about 99.9% by weight of said dichloromethane;
about 0.1% to about 99.9% by weight of said toluene;
about 0.1% to about 99.9% by weight of one of cis-resveratrol, trans-resveratrol, and a combination thereof;
about 0.1% to about 99.9% by weight of said acetic acid;
about 0.1% to about 99.9% by weight of said lauryldimethyl hydroxysultaine; and
a sufficient amount of a base composition to adjust total weight percentage of said chemical composition to 100%.

4. The method of claim 1, wherein said chemical composition comprises one or more additives selected from a group comprising about 0.1% to about 99.9% by weight of said d-limonene, about 0.1% to about 99.9% by weight of monoglyceride, about 0.1% to about 99.9% by weight of monoctanoin, about 0.1% to about 99.9% by weight of diethyl ether, about 0.1% to about 99.9% by weight of halothane, about 0.1% to about 99.9% by weight of ethanol, about 0.1% to about 99.9% by weight of methanol, about 5 mg to about 200 mg of steroids, about 1 mg to about 50 mg of folic acid, about 100 units to about 10000 units of heparin, about 0.1% to about 99.9% by weight of octanediol, about 1 mg to about 50 mg of adenosine, about 0.1% to about 99.9% by weight of high density lipoproteins, about 0.1% to about 99.9% by weight of oils, and any combination thereof and a sufficient amount of a base composition to adjust total weight percentage of said chemical composition to 100%.

5. The method of claim 1, wherein said chemical composition comprises one or more medications comprising one or more of direct thrombin inhibitors, thrombolytics, statins, antiplatelets, calcium channel blockers, and any combination thereof.

6. The method of claim 5, wherein said calcium channel blockers comprise verapamil.

7. The method of claim 1, wherein said chemical composition is administered at said sites of said plaques formed in said blood vessels of said patient through a catheter.

8. The method of claim 1, wherein said administered chemical composition is retained at said sites of said plaques for a predetermined period of time to enable said modification of said plaques before removing said administered chemical composition by one of suctioning and flushing.

9. The method of claim 1, wherein said modification of said plaques by said administered chemical composition comprises one of partial dissolution, complete dissolution and modification of said plaques in coronary arteries for enhancing luminal patency of said coronary arteries.

10. The method of claim 1, wherein said modification of said plaques by said administered chemical composition comprises one of partial dissolution, complete dissolution and modification of said plaques in peripheral arteries and arteries in lower extremities of a body of said patient for enhancing luminal patency of said peripheral arteries and said arteries of said lower extremities.

11. The method of claim 1, wherein said modification of said plaques by said administered chemical composition comprises one of partial dissolution, complete dissolution and modification of said plaques in renal arteries for enhancing luminal patency of said renal arteries.

12. The method of claim 1, wherein said modification of said plaques by said administered chemical composition comprises one of partial dissolution, complete dissolution and modification of said plaques in carotid arteries and cerebral vessels for enhancing luminal patency of said carotid arteries and said cerebral vessels.

13. The method of claim 1, wherein said modification of said plaques by said administered chemical composition comprises partial dissolution of said plaques for creating a channel for blood flow and for passing interventional equipment for said different forms of said plaque treatment.

14. The method of claim 1, further comprising administering adjunctive methods comprising utilizing one or more of low and high temperatures, pressure, radio magnetic waves, and physical agitation to obtain desired modification of said plaques.

15. A chemical composition for modifying and dissolving atherosclerotic plaque in blood vessels of a patient, comprising one or more of:
about 0.1% to about 99.9% by weight of d-limonene;
about 0.1% to about 99.9% by weight of propylene glycol;
about 0.1% to about 99.9% by weight of octanoic acid;
about 0.1% to about 99.9% by weight of 2-octane;
about 0.1% to about 99.9% by weight of glycerine;
about 0.1% to about 99.9% by weight of acetylsalicylic acid;
about 0.1% to about 99.9% by weight of omega-3 fatty acids;
about 0.1% to about 99.9% by weight of ezetimibe;
about 0.1% to about 99.9% by weight of said rosuvastatin;
about 0.1% to about 99.9% by weight of lactic acid;
about 0.1% to about 99.9% by weight of gluconic acid;
about 0.1% to about 99.9% by weight of chloroform;
about 0.1% to about 99.9% by weight of carbon disulfide;
about 0.1% to about 99.9% by weight of dichloromethane;
about 0.1% to about 99.9% by weight of toluene;
about 0.1% to about 99.9% by weight of one of cis-resveratrol, trans-resveratrol, and a combination thereof;
about 0.1% to about 99.9% by weight of acetic acid;
about 0.1% to about 99.9% by weight of lauryldimethyl hydroxysultaine; and
a sufficient amount of a base composition to adjust total weight percentage of said chemical composition to 100%;
whereby said chemical composition administered at sites of said atherosclerotic plaque modifies said atherosclerotic plaque by altering composition of said atherosclerotic plaque formed in said blood vessels.

16. The chemical composition of claim 15, further comprising one or more of about 0.1% to about 99.9% by weight of monoglyceride, about 0.1% to about 99.9% by weight of monoctanoin, about 0.1% to about 99.9% by weight of diethyl ether, about 0.1% to about 99.9% by weight of halothane, about 0.1% to about 99.9% by weight of ethanol, about 0.1% to about 99.9% by weight of methanol, about 5 mg to about 200 mg of steroids, about 1 mg to about 50 mg of folic acid, about 100 units to about 10000 units of heparin, about 0.1% to about 99.9% by weight of octanediol, about 1 mg to about 50 mg of adenosine, about 0.1% to about 99.9% by weight of high density lipoproteins, about 0.1% to about 99.9% by weight of oils, and any combination thereof and a sufficient amount of a base composition to adjust total weight percentage of said chemical composition to 100%.

17. The chemical composition of claim 15, wherein said base composition comprises one of water, dextrose water, a saline solution, and an organic solvent.

18. A chemical composition for modifying and dissolving plaques in blood vessels of a patient, comprising one or more of:
about 0.1% to about 100% by weight of one or more of an organic substance, an inorganic substance, and a bioactive substance, wherein said organic substance, said inorganic substance, and said bioactive substance are selected from a group comprising one or more of d-limonene, propylene glycol, octanoic acid, 2-octane, glycerine, acetylsalicylic acid, acetic acid, omega-3 fatty acid, ezetimibe, rosuvastatin, resveratrol, lactic acid, gluconic acid, chloroform, carbon disulfide, dichloromethane, toluene, lauryldimethyl hydroxysultaine, and any combination thereof; and
about 0% to about 99.9% by weight of one or more additives selected from a group comprising d-limonene, monoglyceride, monoctanoin, diethyl ether, halothane, ethanol, methanol, steroids, folic acid, heparin, octanediol, adenosine, high density lipoproteins, oils, and any combination thereof;
whereby said chemical composition administered at sites of said plaques formed in said blood vessels of said patient modifies said plaques by altering composition of said plaques in said blood vessels.

19. The chemical composition of claim 18, wherein said d-limonene is about 20% to about 100% by weight of said chemical composition.

20. The chemical composition of claim 18, wherein said propylene glycol is about 20% to about 99.9% by weight of said chemical composition.

21. The chemical composition of claim 18, wherein said octanoic acid is about 10% to about 98% by weight of said chemical composition.

22. The chemical composition of claim 18, wherein said 2-octane is about 10% to about 98% by weight of said chemical composition.

23. The chemical composition of claim 18, wherein said glycerine is about 20% to about 100% by weight of said chemical composition.

24. The chemical composition of claim 18, wherein said acetylsalicylic acid is about 0.1% to about 99.9% by weight of said chemical composition.

25. The chemical composition of claim 18, wherein said omega-3 fatty acid is about 0.1% to about 99.9% by weight of said chemical composition.

26. The chemical composition of claim 18, wherein said acetic acid is about 0.1% to about 99.9% by weight of said chemical composition.

27. The chemical composition of claim 18, wherein said ezetimibe is about 0.1% to about 99.9% by weight of said chemical composition.

28. The chemical composition of claim 18, wherein said rosuvastatin is about 0.1% to about 99.9% by weight of said chemical composition.

29. The chemical composition of claim 18, wherein said resveratrol is about 0.1% to about 99.9% by weight of said chemical composition.

30. The chemical composition of claim 18, wherein said lactic acid is about 0.1% to about 99.9% by weight of said chemical composition.

31. The chemical composition of claim 18, wherein said gluconic acid is about 0.1% to about 99.9% by weight of said chemical composition.

32. The chemical composition of claim 18, wherein said chloroform is about 0.1% to about 99.9% by weight of said chemical composition.

33. The chemical composition of claim 18, wherein said carbon disulfide is about 0.1% to about 99.9% by weight of said chemical composition.

34. The chemical composition of claim 18, wherein said dichloromethane is one of with methanol and without methanol.

35. The chemical composition of claim 18, wherein said dichloromethane is about 0.1% to about 99.9% by weight of said chemical composition.

36. The chemical composition of claim 18, wherein said toluene is about 0.1% to about 99.9% by weight of said chemical composition.

37. The chemical composition of claim 18, wherein said lauryldimethyl hydroxysultaine is about 0.1% to about 99.9% by weight of said chemical composition.

38. The chemical composition of claim 18, wherein said one or more additives are selected from a group comprising about 0.1% to about 99.9% by weight of said d-limonene, 0.1% to about 99.9% by weight of said monoglyceride, about 0.1% to about 99.9% by weight of said monoctanoin, about 1% to about 20% by weight of said diethyl ether, about 0.1% to about 99.9% by weight of said halothane, about 0.1% to about 99.9% by weight of said ethanol, about 0.1% to about 99.9% by weight of said methanol, about 5 mg to about 200 mg of said steroids, about 1 mg to about 50 mg of said folic acid, about 100 units to about 10000 units of said heparin, about 0.1% to about 99.9% by weight of said octanediol, about 1 mg to about 50 mg of said adenosine, about 0.1% to about 99.9% by weight of said high density lipoproteins, about 0.1% to about 99.9% by weight of said oils, and any combination thereof.

39. The chemical composition of claim 18, further comprising one or more optional medications comprising one or more of direct thrombin inhibitors, thrombolytics, statins, antiplatelets, calcium channel blockers, and any combination thereof.

40. The chemical composition of claim 18, further comprising a sufficient amount of a base composition to adjust total weight percentage of said chemical composition to 100%, wherein said base composition comprises one of water, dextrose water, a saline solution, an organic solvent, and any combination thereof.
